(19) United States
(12) Patent Application Publication
(10) Pub. No.: US 2025/0375359 A1
(43) Pub. Date: Dec. 11, 2025

(54) COMPOSITION THAT DRIES TO POWDER, METHODS OF MAKING, AND PERSONAL USE OF SAME

(71) Applicant: DR. SQUATCH, LLC, MARINA DEL REY, CA (US)

(72) Inventors: Alexander Uruchurtu, Los Angeles, CA (US); Mason Montgomery, Inglewood, CA (US); Sudheer Koneru, Austin, TX (US); Monica Contreras Medellin, Austin, TX (US); Tobie Spitzer, La Habra, CA (US); Alexandra Blackstone, MARINA DEL REY, CA (US); Duane Kleingarn, MARINA DEL REY, CA (US)

(73) Assignee: DR. SQUATCH, LLC, MARINA DEL REY, CA (US)

(21) Appl. No.: 19/230,849

(22) Filed: Jun. 6, 2025

Related U.S. Application Data
(60) Provisional application No. 63/657,674, filed on Jun. 7, 2024.
(60) Provisional application No. 63/759,757, filed on Feb. 18, 2025.

(51) Int. Cl. A61K 8/02; A61Q 15/00

(52) U.S. Cl. CPC A61K 8/0204; A61Q 15/00; A61K 2800/34; A61K 2800/805; A61K 2800/85

(57) ABSTRACT

Precision deodorant compositions are disclosed herein that are applied as a lotion and quickly dry to a powder. The compositions include a starch, an alcohol as a solvent, water, ethyl macadamiate and/or methylheptylglycerin. The percent wt/wt of water is greater than the percent wt/wt of starch and is greater than the percent wt/wt of alcohol, and the emollient and deodorant agent are natural ingredients. The composition is substantially free of silicones, coconut oil, fractionated coconut oil, and phenoxyethanol.

### TECHNICAL FIELD

This application relates to compositions that dry to a powder form, and more particularly to deodorant compositions that are topically applied to skin and dry to a powder or powder-like consistency or feel and include water, alcohol, starch, and a selection of natural ingredients, including a natural deodorant agent.

### BACKGROUND

Powders (dry dusting powders) are useful for many applications, such as to keep a baby's bottom dry or as a personal hygiene aide. Powders are messy when sprinkling or “dusting” a surface. The loose powder tends to fall over a wide area including the floor, countertop, or a user's clothes.

There is a need to be able to achieve a powdered surface while overcoming the above issue, especially with a composition made of natural ingredients including a natural deodorant agent.

### SUMMARY

In one aspect, topical compositions such as a hygiene composition, a cosmetic composition, or a dry lotion deodorant that dry to a powder form are disclosed that include a starch, an alcohol as a solvent, water, an emollient, and a deodorant agent. The % wt/wt water is greater than the % wt/wt starch and the % wt/wt alcohol. The topical composition is free or substantially free of silicone(s), coconut oil, fractionated coconut oil, and phenoxyethanol, and the emollient and deodorant agent are natural or naturally-derived ingredients. In one embodiment, the water is greater than 35% wt/wt of the composition, the starch is less than 35% wt/wt of the composition and the denatured alcohol is less than 20% wt/wt of the composition. In another embodiment, the water is greater than 40% wt/wt of the composition, the starch is less than 35% wt/wt of the composition, and the denatured alcohol is less than 15% wt/wt of the composition. In a third embodiment, the water is greater than 45% wt/wt of the composition, the starch is less than 30% wt/wt of the composition, and the denatured alcohol is less than 15% wt/wt of the composition. In a fourth embodiment, the water is greater than 50% wt/wt of the composition, the starch is less than 35% wt/wt of the composition, and the denatured alcohol is less than 15% wt/wt of the composition. In all aspects, the topical composition can be a cream, a lotion, or a serum.

In all aspects, the alcohol can be a denatured alcohol having 1 to 4 carbons. In all aspects, the deodorant agent can include methylheptylglycerin and/or Saccharomyces ferment filtrate. In all aspects, the emollient can include ethyl macadamiate and can be about 2% wt/wt to about 10% wt/wt of the composition. Additionally, the emollient can include a mixture of hydrogenated ethylhexyl olivate and hydrogenated olive oil unsaponifiables. When this mixture is present, the hydrogenated ethylhexyl olivate has a % wt/wt greater than the % wt/wt of the hydrogenated olive oil unsaponifiables.

In all formulations, the topical composition can include a skin conditioning agent as about 0.05% wt/wt to about 3% wt/wt of the composition. The skin conditioning agent can include a mixture of Aloe Barbadensis leaf juice and sunflower seed oil. The skin conditioning agent can include shea butter.

In all formulations, a super absorbent polymer can be present and can be about 0.01% to about 5% of the composition. The super absorbent polymer can include sodium polyacrylate.

In some embodiments, the starch is a mixture of tapioca starch and arrowroot powder. Some embodiments include a fragrance in a range of about 0.1% wt/wt to about 6% wt/wt of the composition, and, optionally, include less than 1% wt/wt of other ingredients selected from the group consisting of aloe Barbadensis leaf juice, hydrogenated olive oil unsaponifiables, tocopherol, sunflower seed oil, shea butter, potassium sorbate, sodium benzoate and combinations thereof.

### BRIEF DESCRIPTION OF THE DRAWING

FIG. 1 is a flow chart of a method of making the compositions disclosed herein.

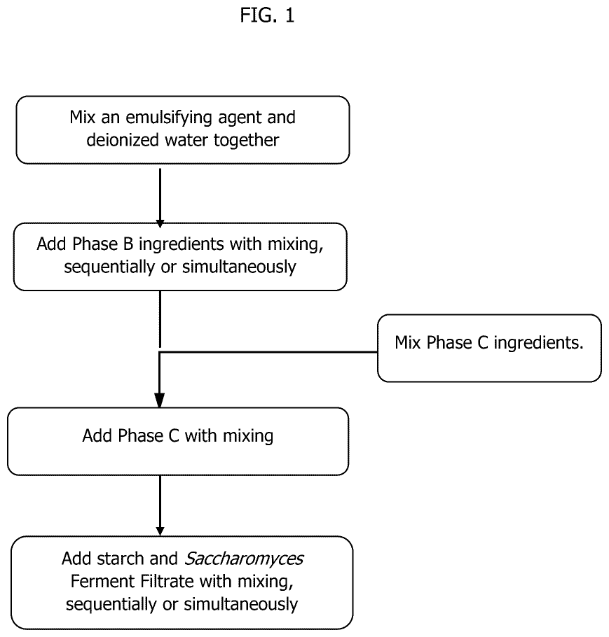

FIG. 2 is a chart of data from an In-Home Use Test with 105 participants.

### DETAILED DESCRIPTION

In all aspects, compositions that dry to form a powder are disclosed. The compositions provide a clean application to the intended surface because dry powder does not escape and fall over a broad area beyond the intended surface. Yet, once dry, the composition provides the same feeling as a powder. Moreover, the composition utilizes natural ingredients and is optionally fragranced.

As used herein, a natural ingredient is an ingredient obtained only from a natural source such as a plant, animal, micro-organism, or mineral; however, ingredients obtained from fossil fuels are outside the definition of a natural ingredient. Natural ingredients include ingredients obtained from natural sources through physical processes, fermentation reactions occurring in nature and leading to molecules which occur in nature, and other procedures of preparation that do not involve intentional chemical modification. (International Organization for Standardization (ISO) 16128-1). A naturally derived ingredient is an ingredient of greater than 50% natural origin, by molecular weight, by renewal carbon content, or by other methods used in the art, obtained through chemical and/or biological processes resulting in chemical modification. (ISO 16128-1). Chemical modifications falling within the scope of naturally derived ingredient include, for example, saponification, neutralization, hydration, etherification, amination, esterification, amidation, and hydrolysis. The relevant chemical structure of the natural ingredient remaining intact means, by way of example, that the fatty chain acyl structure of a fatty acid remains after esterification or amidation. The above is consistent with the definition of “natural” as calculated by the ISO 16128-1 and/or ISO 16128-2 methodologies/mathematical formulations set forth therein. (ISO 16128-1 (2016) Guidelines on technical definitions and criteria for natural and organic cosmetic ingredients and products—Part 1: Definitions for ingredients, and ISO 16128-2 (2017) Guidelines on technical definitions and criteria for natural and organic cosmetic ingredients and products—Part 2: Criteria for ingredients and products, both of which are hereby incorporated herein by reference in their entirety.)

“Natural” or “naturally-derived” is applied herein to substances, ingredients, and products, and may be collectively referred to as natural substances, natural ingredients, and natural products. In other words, the use of the phrase “natural” herein is understood to mean natural and/or naturally derived in all instances. The natural content of a formulation can be calculated based on the ISO methodologies noted above, especially ISO 16128-2 (natural origin). Each ingredient, taken separately, meets the definition of a naturally-derived ingredient if it is an ingredient having greater than 50% natural origin. The natural content of a formulation is the mass percentage of all natural or naturally-derived ingredients present therein. It is calculated as the sum of the relative concentrations of each ingredient in the formulation multiplied by each ingredient's respective percentage of natural origin. Again, this is explained in detail in ISO 16218-2 (5.2). Each deodorant composition disclosed herein as a whole is composed of at least 95% natural ingredients, more preferably at least 98% natural ingredients.

All numerical values set forth herein can be modified by the word “about” whether expressly stated or not. “About” as used herein means plus or minus 5% of a numerical value, or more preferably plus or minus 2%. For ranges, the minimum and maximum values are included in the range and all individual values therebetween. It is further understood that the endpoints of each range are significant both in relation to the other endpoint, and independently of the other endpoint. Percentages for concentrations are typically % by weight/weight unless expressly stated otherwise. “Substantially free” means 1% wt/wt or 0.5% wt/wt, or more preferably 0.01% wt/wt or less are present of the identified substance.

In all aspects, compositions are disclosed herein that after application dry to a powder form. As such, the compositions have a high payload of powder in a cosmetically or biologically suitable carrier that will evaporate to deposit the powder. The compositions are natural compositions, i.e., being greater than 95% natural, preferably at or greater than 98% natural per ISO 16128-1 and/or ISO 16128-2. It is understood that each ingredient discussed below is a natural ingredient, even if not expressly stated again. The compositions can be a topical composition such as a hygiene composition, a cosmetic composition, or a dry lotion deodorant (also called a precision deodorant because of the ease of application on any portion of the body).

The topical compositions can be formulated as a cream or crème, a gel-crème, a gel, a lotion, a serum, with the order from cream to serum being thicker to thinner (i.e., higher viscosity to lower viscosity), or other flowable material. Viscosity can be measured using a viscometer or flow cups. A Viscometer measures the resistance of a liquid to flow, providing a quantitative value for viscosity. Any commercially available viscometer can be used including capillary, rotational, and oscillatory viscometers using known procedures or a standardized procedure. Flow cups measure the time it takes for a liquid to flow through a hole at the bottom of a cup. This method provides a quick estimate of viscosity, which may be useful for formulations of low to moderate viscosity.

In the formulations disclosed herein the viscosity of the composition can be adjusted by changing the concentration of one or more substances therein, such as starch, water, alcohol, waxes, and super absorbent polymer, with the waxes having the least amount of flexibility to its concentration. In one embodiment, to reduce the viscosity, the concentration of the starch can decrease as the concentration of water is increased. In another embodiment, the concentration of the alcohol can be increased alone or in correlation to the concentration of the starch. In yet another embodiment, the amount of super absorbent polymer present can be changed. For a cream, the concentration of super absorbent polymer can be at or greater than 2% wt/wt of the composition. For a lotion, the concentration of super absorbent polymer can be at or greater than 1% to 2% wt/wt of the composition. For a serum, the concentration of super absorbent polymer can be at or less than 1% wt/wt of the composition.

The powder is typically a starch but could be or include other absorbent powders or a blend of absorbent powders and starches. The starch can be selected from the group consisting of corn starch, wheat starch, rice starch, potato starch, tapioca starch, arrowroot starch (often called arrowroot powder), and combinations thereof. Arrowroot powder may be from any one of the following plants Maranta arundinacea, Zamia integrifolia, Manihot esculenta, Tacca leontopetaloides, Pueraria lobata. In all embodiments, the composition can include a mixture of starches. One example of a mixture of starches includes tapioca starch and arrowroot powder. Examples of other absorbent powders, includes but is not limited to, silica, charcoal, talc, silica-derivatives, natural clays, and natural minerals. Some examples of natural clays include bentonite or kaolin clay. In some embodiments, the tapioca starch is present in a greater % wt/wt than the arrowroot powder. The ratio of arrowroot powder to tapioca starch can be in a range of 1:10 to 1:30 based on the % wt/wt values.

The suitable carrier that will evaporate to deposit the powder includes water, typically deionized water and an alcohol. The carrier can be an emulsified water base mixed with water soluble ingredients and with an alcohol phase. The alcohol phase can have an alcohol or a denatured alcohol and one or more emollients and skin conditioning agents, and optionally a fragrance. The powder is loaded into this mixture of emulsified water and alcohol phase to provide a composition. The composition includes a denatured alcohol. The alcohol is a short chain alcohol, one having 1-4 carbons, such as methanol, ethanol, isopropanol, derived from grain. SD-Alcohol and some “number+letter” refers to ethanol being denatured with different kind of additives. 40-B means the additives are denatonium benzoate and t-butyl alcohol. The alcohol is a natural product and aids in quick drying of the composition and can be a solvent for some of the ingredients.

The composition has a starch, a denatured alcohol comprising 1, 2, 3, or 4 carbons, water, an emollient, a skin conditioning agent, a deodorant agent, and optionally a fragrance. The composition has a % wt/wt water, which is greater than a % wt/wt starch and is greater than a % wt/wt of the denatured alcohol. The water is equal to or greater than 35% wt/wt, or equal to or greater than 40% wt/wt, or equal to or greater than 45% wt/wt, or equal to or greater than 50% wt/wt of the composition. The denatured alcohol is 30% wt/wt or less, or 20% wt/wt or less, or 15% wt/wt or less of the composition. The starch is 35% wt/wt or less, or 30% wt/wt or less of the composition.

In one embodiment, the water is greater than 35% wt/wt of the composition, the starch is less than 35% wt/wt of the composition and the denatured alcohol is less than 20% wt/wt of the composition. In another embodiment, the water is greater than 40% wt/wt of the composition, the starch is less than 35% wt/wt of the composition, and the denatured alcohol is less than 15% wt/wt of the composition. In yet another embodiment, the water is greater than 45% wt/wt of the composition, the starch is less than 30% wt/wt of the composition, and the denatured alcohol is less than 15% wt/wt of the composition. And, in yet another embodiment, the water is greater than 50% wt/wt of the composition, the starch is less than 35% wt/wt of the composition, and the denatured alcohol is less than 15% wt/wt of the composition.

In all embodiments, the composition can include a deodorant agent, which can be odor-eliminating and/or antimicrobial ingredients. Suitable natural deodorant agents include methylheptylglycerin, Saccharomyces ferment filtrate, zinc derivatives, triethyl citrate, and polyglyceryl-3 caprylate. Some non-limiting examples of zinc derivatives include zinc ricinoleate and zinc PCA (a zinc salt of pyrrolidone carboxylic acid). The composition can be one or a mixture of any number of deodorant agents. Methylheptylglycerin prevents malodor through the inhibition of the growth of malodor causing bacteria. Methylheptylglycerin is available from Inolex, Inc. under the mark LEXGARD® MHG Natural MB. Saccharomyces ferment filtrate, a natural ingredient obtained by fermenting and filtering yeasts can soothe, hydrate, and protect skin and hair. This functional deodorizing agent uses enzymes created by the fermentation of natural sugar cane or rice. Other post-bacterial ferments can be used as well if they have suitable deodorizing properties. DEOPLEX® natural deodorant is available from Carrubba, Inc. The compositions are free of aluminum-containing compounds. The deodorant agent may include baking soda and/or magnesium-containing compounds, but the example formulations herein are free or substantially free thereof. Some examples of magnesium-containing compounds include magnesium hydroxide, magnesium carbonate, magnesium carbonate hydroxide, and combinations thereof.

In all embodiments, the composition can include a super absorbent polymer, especially as an emulsifying agent for the water phase. Super absorbent polymers can be cross-linked polyacrylates and polyacrylamides, cellulose- or starch-acrylonitrile graft copolymers, cross-linked maleic anhydride copolymers, and/or hemicellulose plant polysaccharides. Examples include polyacrylamide, sodium polyacrylate, absorbent polysaccharide derived polymers such as xanthan gum and gellan gum, and hemicelluloses such as xylan, glucuronoxylan, arabinoxylan, mannan, glucomannan, and galactoglucomannan. The super absorbent polymer can be present in a range from 0.01% to about 5% wt/wt of the composition, preferably about 0.1% to about 2% wt/wt of the composition. For a lotion, the super absorbent polymer is at most about 1% wt/wt of the composition.

In all embodiments, the composition includes an emollient and can include a plurality of emollients. The emollient comprises about 2% wt/wt to about 10% wt/wt of the composition. Natural emollients may be plant or animal based butters, oils, or waxes. Botanical butters are often made from seeds, pips, or nuts of the plant. Some nonlimiting examples of botanical butters include: Bacuri butter, cocoa butter, mango butter, and shea butter. Some nonlimiting examples of plant derived oils include: acai oil, almond oil, apricot oil, avocado oil, blackberry oil, black cumin seed oil, Brazil nut oil, calendula oil, camellia oil, canola oil, castor oil, coconut oil, corn oil, cranberry oil, cucumber seed oil, hazel nut oil, hemp seed oil, jojoba oil, kiwi oil, macadamia nut oil, olive oil, hydrogenated olive oil unsaponifiables, palm oil, palm kernel oil, papaya oil, passion fruit oil, primrose oil, rice bran oil, rosehip oil, sunflower seed oil, watermelon seed oil, and wheat germ oil. Some nonlimiting examples of plant derived waxes include sunflower wax, candelilla wax, carnauba wax, bayberry wax, castor wax (also known as hydrogenated castor oil), beeswax, rapeseed wax, rice bran wax, and combinations thereof. It is also possible to include fatty acid based waxes such as fatty alcohols, fatty acid esters (ester waxes), or fatty acids.

Additionally, the emollient may be a subcomponent of or derived from a plant or animal-based butter, oil, or wax. In one embodiment, the emollient is selected from the group consisting of ethyl macadamiate, hydrogenated ethylhexyl olivate, hydrogenated olive oil unsaponifiables, and mixtures thereof. In one embodiment, the emollient includes ethyl macadamiate. Ethyl macadamiate is the ester of ethyl alcohol mixed with fatty acids from macadamia nut oil to create an emollient, which is available under the brand name FLORAMAC® 10 ester from Cargill, Inc. On skin, ethyl macadamiate softens, conditions, and prevents moisture loss. In another embodiment, the emollient includes a mixture of hydrogenated ethylhexyl olivate and hydrogenated olive oil unsaponifiables and can also include ethyl macadamiate. When the composition includes a mixture of hydrogenated ethylhexyl olivate and hydrogenated olive oil unsaponifiables, the hydrogenated ethylhexyl olivate is present in a % wt/wt that is greater than the % wt/wt of the hydrogenated olive oil unsaponifiables. The hydrogenation process to make hydrogenated ethylhexyl olivate involves the addition of hydrogen molecules to transform a liquid into a solid or semi-solid state. Ethylhexyl olivate itself is an ester of skin-conditioning fatty acids derived from olive oil. Like its liquid form, hydrogenated ethylhexyl olivate functions as an emollient and can function as a skin conditioning agent. A mixture of hydrogenated ethylhexyl olivate and hydrogenated olive oil unsaponifiables is commercially available under the brand name PLANTASENS® Olive LD from Clariant, Ltd.

In all embodiments, the compositions can include a skin conditioning agent or a plurality of skin conditioning agent. The compositions can include the skin conditioning agent as about 0.05% wt/wt to about 3% wt/wt of the composition. The botanical butters noted above as emollients may also be considered here as a skin conditioning agent. Some of the plant based or derived oils may also be skin conditioning agents, such as sunflower seed oil. Another plant based skin conditioning agent is Aloe Barbadensis leaf juice. In one embodiment, the skin conditioning agent comprises a mixture of Aloe Barbadensis leaf juice and sunflower seed oil. The skin conditioning agent can also include butter.

In all embodiments, the compositions can include a chelating agent added to the water phase. Suitable examples of chelating agents, include but are not limited to, phytic acid, sodium phytate, ethylenediaminetetraacetic acid (EDTA), ethylenediamine-N, N′-disuccinic acid (EDDS), amino acid based chelating agents such as tetrasodium glutamate diacetate, and caprylhydroxamic acid. In one embodiment, the chelating agent is sodium phytate. The chelating agent binds certain metals present with water, and it can function as a stabilizer. The chelating agent can be present as less than 1% wt/wt of the composition, more preferably less than 0.5% wt/wt of the composition. In one embodiment, the chelating agent is present as 0.1% wt/wt of the composition.

The compositions can include other ingredients selected from the group consisting of vitamins, preservatives, coloring agents, anti-whitening agents, fragrances, pH adjusters, skin-benefit agents, etc. Some examples of skin benefits include moisturizing, tightening, conditioning, softening, protecting, and the like. Preferably, the composition is made 100% of natural or naturally derived ingredients or is composed of at least 95% or more (as a whole) natural or naturally derived ingredients. In another embodiment, the composition is at least composed of 98% natural or naturally derived ingredients. In one embodiment, the pH adjuster is present and is less than 0.05% wt/wt of the composition.

The compositions disclosed herein can include less than 1% wt/wt each of the following: Aloe Barbadensis leaf juice, hydrogenated olive oil unsaponifiables, tocopherol, sunflower seed oil, shea butter, potassium sorbate, sodium benzoate and combinations thereof.

In one embodiment, the composition includes a starch or a plurality of starches, a denatured alcohol comprising 1 to 4 carbons, water, and ethyl macadamiate. The % wt/wt of the water is greater than the % wt/wt of the starch and is greater than the % wt/wt of the denatured alcohol. The composition is substantially free of silicone(s) and dries to a powder form. The water is equal to or greater than 35% wt/wt, or equal to or greater than 40% wt/wt, or equal to or greater than 45% wt/wt, or equal to or greater than 50% wt/wt of the composition. The denatured alcohol is 30% wt/wt or less, or 20% wt/wt or less, or 15% wt/wt or less of the composition. The starch is 35% wt/wt or less, or 30% wt/wt or less of the composition.

In one embodiment, the composition includes a starch or a plurality of starches, a denatured alcohol comprising 1 to 4 carbons, water, and methylheptylglycerin. The % wt/wt of the water is greater than the % wt/wt of the starch and is greater than the % wt/wt of the denatured alcohol. The composition is substantially free of silicone(s) and dries to a powder form. The water is equal to or greater than 35% wt/wt, or equal to or greater than 40% wt/wt, or equal to or greater than 45% wt/wt, or equal to or greater than 50% wt/wt of the composition. The denatured alcohol is 30% wt/wt or less, or 20% wt/wt or less, or 15% wt/wt or less of the composition. The starch is 35% wt/wt or less, or 30% wt/wt or less of the composition.

### Example 1

TABLE 1

| | Percentage Range | Percentage Range |
|---|---|---|
| | All Formulations | Lotions |
| INCI | (% wt/wt) | (% wt/wt) |
| Water | 30-85 | 30-60 |
| Tapioca Starch | 5-30 | 10-30 |
| SD Alcohol 40-B | 5-30 | 10-30 |
| Ethyl Macadamiate | 3-10 | 3-10 |
| Saccharomyces Ferment Filtrate | 1-3 | 1-3 |
| Methylheptylglycerin | 1-3 | 1-3 |
| Maranta Arundinacea (Arrowroot) Root Powder | 1-3 | 1-3 |
| Hydrogenated Ethylhexyl Olivate | 0.3-1 | 0.3-1 |
| Super absorbent polymer | 0.1-5 | 0.3-1 |
| Sodium Phytate | 0.1-0.3 | 0.1-0.3 |
| Aloe Barbadensis Leaf Juice | 0-0.1 | 0-0.1 |
| Hydrogenated Olive Oil Unsaponifiables | 0-0.1 | 0-0.1 |
| Tocopherol | 0-0.1 | 0-0.1 |
| Helianthus Annuus (Sunflower) Seed Oil | 0-0.1 | 0-0.1 |
| Butyrospermum Parkii (Shea) Butter | 0-0.1 | 0-0.1 |
| Potassium Sorbate | 0-0.1 | 0-0.1 |
| Sodium Benzoate | 0-0.1 | 0-0.1 |

More specifically, those substances in Table 1 above that comprise 0.1% or less of the composition may be present as:

TABLE 2

| INCI | % wt/wt |
|---|---|
| Aloe Barbadensis Leaf Juice | 0.001-0.1 |
| Hydrogenated Olive Oil Unsaponifiables | 0.001-0.1 |
| Tocopherol | 0.001-0.1 |
| Helianthus Annuus (Sunflower) Seed Oil | 0.001-0.1 |
| Butyrospermum Parkii (Shea) Butter | 0.001-0.1 |
| Potassium Sorbate | 0-0.1 |
| Sodium Benzoate | 0-0.1 |

### Working Examples

TABLE 3
Lotion Deodorants

| | | Example 2 | Example 3 |
|---|---|---|---|
| | | Concentration | Concentration |
| Phases | INCI | (% wt/wt) | (% wt/wt) |
| Phase A | | | |
| | Water | 52.7 | 53.4 |
| | Sodium Polyacrylate | 0.8 | 0.8 |
| Phase B | | | |
| | Methylheptylglycerin | 1 | 1 |
| | Sodium Phytate | 0.1 | 0.1 |
| | Aloe Barbadensis Leaf Juice | 0.05 | 0.05 |
| Phase C | | | |
| | SD Alcohol 40-B | 10 | 10 |
| | Ethyl Macadamiate | 3 | 3 |
| | Hydrogenated Ethylhexyl Olivate | 0.95 | 0.95 |
| | Hydrogenated Olive Oil Unsaponifiables | 0.05 | 0.05 |
| | Tocopherol | 0.05 | 0.05 |
| | Helianthus Annuus (Sunflower) Seed Oil | 0.05 | 0.05 |
| | Butyrospermum Parkii (Shea) Butter | 0.05 | 0.05 |
| | Fragrance | 0.7 | — |
| Phase D | | | |
| | Saccharomyces Ferment Filtrate | 2 | 2 |
| | Tapioca Starch | 27.5 | 27.5 |
| | Maranta Arundinacea (Arrowroot) Root | 1 | 1 |
| | Powder | | |

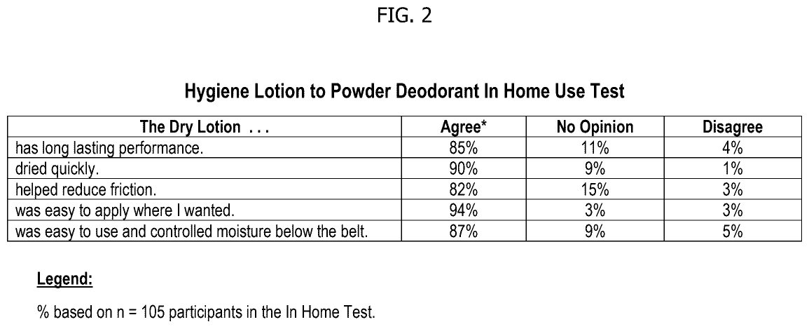

Example 2 was used for an In Home Test study, which is reported in FIG. 2. One hundred and five users tested a precision lotion deodorant and answered the questionnaire regarding their experience. Ninety four percent of the participants were of the option that the precision lotion deodorant was easy to apply where wanted and ninety percent agreed that it dried quickly. Moreover, eighty seven percent of the participants felt that the precision lotion deodorant controlled moisture below the belt, with eighty five percent agreeing that it had long lasting performance.

With reference to TABLE 1 and the figure, the compositions disclosed herein are made by mixing an emulsifying agent with deionized water and adding the Phase B substances, the emollients, sequentially or simultaneously to form an aqueous solution. Sequential mixing may be preferable. The Phase C substances are mixed sequentially or simultaneously in a separate vessel before being mixed with the aqueous solution. The mixed Phase C is added to the aqueous solution with mixing, then the Phase D substances are added with mixing either sequentially or simultaneously, until the powders are dispersed. Sequential mixing may be preferable.

The lotion composition can optionally include a fragrance. The fragrance can be present in an amount of 0.1% wt/wt to 6% wt/wt, more preferably 0.3% wt/wt to 3% wt/wt, and in some embodiments as 0.3% wt/wt to 1% by wt/wt. In some embodiments, the fragrance comprises about 0.1% wt/wt to about 1% wt/wt of the lotion composition.

In all aspects, the compositions are either free of silicone(s) or substantially free of silicone(s). In all embodiments, the compositions are either free of microspheres or substantially free of microspheres. Microspheres are described in U.S. Pat. No. 6,458,372. In all aspects, the compositions are either free or substantially free of petrolatum, petroleum jelly, parabens, coconut oil, fractionated coconut oil (caprylic/capric triglycerides), sodium chloride, phenoxyethanol, ethylhexylglycerin, propylene glycol, anhydroxylitol, hydroxyacetophenone, and betaglucan.

It should be noted that the embodiments are not limited in their application or use to the details of construction and arrangement of parts and steps illustrated in the drawings and description. Features of the illustrative embodiments, constructions, and variants may be implemented or incorporated in other embodiments, constructions, variants, and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention. Having described the invention in detail and by reference to preferred embodiments thereof, it will be apparent that modifications and variations are possible without departing from the scope of the invention which is defined in the appended claims.

What is claimed is:

1. A topical lotion deodorant comprising
a starch;
an alcohol comprising 1, 2, 3, or 4 carbons;
water; and
ethyl macadamiate and/or methylheptylglycerin;
wherein the % wt/wt of the water is greater than the % wt/wt of the starch and is greater than the % wt/wt of the alcohol, and the topical lotion deodorant is substantially free of silicones;
wherein the topical lotion deodorant dries to a powder form after being topically applied by a user.

2. The topical lotion deodorant of claim 1, wherein the water is greater than 35% wt/wt of the topical lotion deodorant, the starch is less than 35% wt/wt of the topical lotion deodorant and the denatured alcohol is less than 20% wt/wt of the topical lotion deodorant.

3. The topical lotion deodorant of claim 1, wherein the water is greater than 40% wt/wt of the composition, the starch is less than 35% wt/wt of the composition, and the denatured alcohol is less than 15% wt/wt of the composition.

4. The topical lotion deodorant of claim 1, wherein the water is greater than 45% wt/wt of the topical lotion deodorant, the starch is less than 30% wt/wt of the topical lotion deodorant, and the denatured alcohol is less than 15% wt/wt of the topical lotion deodorant.

5. The topical lotion deodorant of claim 1, wherein the water is greater than 50% wt/wt of the topical lotion deodorant, the starch is less than 35% wt/wt of the topical lotion deodorant, and the denatured alcohol is less than 15% wt/wt of the topical lotion deodorant.

6. The topical lotion deodorant of claim 1, further comprising Saccharomyces ferment filtrate.

7. The topical lotion deodorant of claim 1, wherein the ethyl macadamiate comprises about 2% wt/wt to about 10% wt/wt of the topical lotion deodorant.

8. The composition of claim 7, further comprising a mixture of hydrogenated ethylhexyl olivate and hydrogenated olive oil unsaponifiables.

9. The composition of claim 8, wherein the hydrogenated ethylhexyl olivate is present in a % wt/wt that is greater than the % wt/wt of the hydrogenated olive oil unsaponifiables.

10. The composition of claim 1, further comprising a skin conditioning agent as about 0.05% wt/wt to about 3% wt/wt of the composition.

11. The composition of claim 10, wherein the skin conditioning agent comprises a mixture of Aloe Barbadensis leaf juice and sunflower seed oil.

12. The composition of claim 11, wherein the skin conditioning agent further comprises shea butter.

13. The composition of claim 1, further comprising at most about 1% wt/wt of a super absorbent polymer.

14. The composition of claim 13, wherein the super absorbent polymer comprises sodium polyacrylate.

15. The composition of claim 1, wherein the starch comprises a mixture of tapioca starch and arrowroot powder.

16. The composition of claim 1, further comprising a fragrance as about 0.1% wt/wt to about 1% wt/wt of the lotion composition.

17. The composition of claim 1, further comprising:

| | % wt/wt |
|---|---|
| Water | 30-60 |
| Tapioca Starch | 10-30 |
| SD Alcohol 40-B | 10-30 |
| Ethyl Macadamiate | 3-10 |
| Saccharomyces Ferment Filtrate | 1-3 |
| Methylheptylglycerin | 1-3 |
| Maranta Arundinacea (Arrowroot) Root Powder | 1-3 |
| Hydrogenated Ethylhexyl Olivate | 0.3-1 |
| Sodium Polyacrylate | 0.3-1 |
| Sodium Phytate | 0.1-0.3 |

wherein the topical lotion deodorant dries to a powder form.

18. The topical lotion deodorant of claim 17, further comprising less than 0.05% wt/wt of a pH adjuster.

19. The topical lotion deodorant of claim 17, being free of silicones, coconut oil, fractionated coconut oil, and phenoxyethanol.

20. The topical lotion deodorant of claim 17, further comprising:

| | % wt/wt |
|---|---|
| Aloe Barbadensis Leaf Juice | 0.001-0.1 |
| Hydrogenated Olive Oil Unsaponifiables | 0.001-0.1 |
| Tocopherol | 0.001-0.1 |
| Helianthus Annuus (Sunflower) Seed Oil | 0.001-0.1 |
| Butyrospermum Parkii (Shea) Butter | 0.001-0.1 |
| Potassium Sorbate | 0-0.1 |
| Sodium Benzoate | 0-0.1 |
